(19) United States
(12) Patent Application Publication
(10) Pub. No.: US 2018/0105481 A1
(43) Pub. Date: Apr. 19, 2018

(54) BIO-BASED METHACRYLIC ACID AND OTHER ALKENOIC-DERIVED MONOMERS VIA CATALYTIC DECARBOXYLATION

(71) Applicant: The United States of America, as represented by the Secretary of Agriculture, Washington, DC (US)

(72) Inventors: Bryan R. Moser, Morton, IL (US); James C. Lansing, Peoria, IL (US); Rex E. Murray, Peoria, IL (US)

(21) Appl. No.: 15/723,432

(22) Filed: Oct. 3, 2017

Related U.S. Application Data
(60) Provisional application No. 62/408,169, filed on Oct. 14, 2016.

(51) Int. Cl. C07C 51/38

(52) U.S. Cl. CPC C07C 51/38

(57) ABSTRACT

A novel method for the catalytic selective decarboxylation of a starting material to produce an organic acid is disclosed. According to at least one embodiment, the method may include placing a reaction mixture into a reaction vessel, the reaction mixture including a solvent, a starting material, and a catalyst, subjecting the reaction mixture to a predetermined pressure and temperature, and allowing the reaction to continue for 1-3 hours. The starting material may be at least one of a dicarboxylic acid, a tricarboxylic acid, and an anhydride of a dicarboxylic or tricarboxylic acid. As an exemplary embodiment, itaconic acid may be a starting material and the organic acid may be methacrylic acid. The predetermined temperature may be 250° C. or less, and the reaction pressure may be less than 425 psi. Further, a polymerization inhibitor may be used.

### REFERENCE TO RELATED APPLICATIONS

This application claims the benefit of U.S. Provisional Application No. 62/408,169, filed Oct. 14, 2016, which is incorporated herein by reference in its entirety.

### BACKGROUND

Methacrylic acid (2-methylpropenoic acid; MAA) and its corresponding esters are important commodity monomers for production of numerous industrially significant plastics. Methyl methacrylate (MMA) is the most useful of these esters and its principal application is homopolymerization to poly(methyl methacrylate) (Plexiglas®) (PMMA), which is a transparent, lightweight and shatter-resistant thermoplastic that is a versatile alternative to glass. MMA is also an essential component of copolymers used for surface coatings, paints, adhesives, and emulsion polymers. The estimated worldwide market for MAA/MMA is in excess of nine billion US dollars per annum.

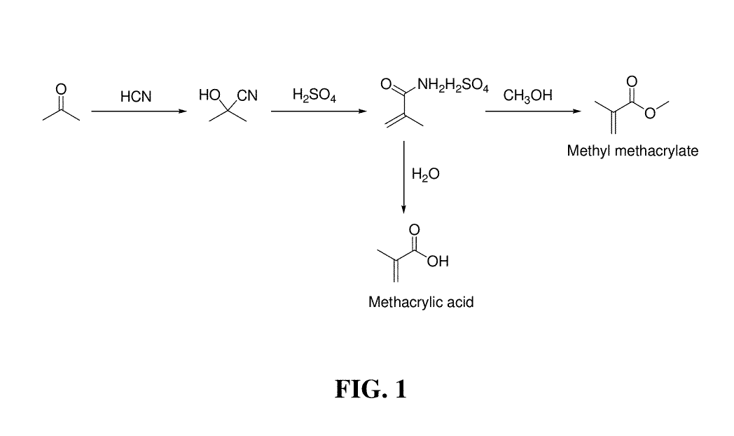

The most significant commercial petrochemical route to MAA is the acetone cyanohydrin process (ACH) depicted in FIG. 1. Acetone reacts with hydrogen cyanide to form an acetone cyanohydrin intermediate, which is readily converted to a methacrylamide sulfate salt upon treatment with stoichiometric concentrated sulfuric acid at 140° C. The intermediate sulfate salt is then converted to either MAA or MMA by reaction with water or anhydrous methanol, respectively. Along with production of MAA/MMA, a stoichiometric excess of ammonium bisulfate byproduct is obtained in a molar ratio of 1.5:1. However, the ACH process utilizes nonrenewable starting materials, stoichiometric amounts of harmful, toxic and corrosive reagents, generates toxic intermediates, and produces a molar excess of low-value ammonium bisulfate byproduct, which must be further reacted with ammonia to yield ammonium sulfate for use as a fertilizer. In particular, hydrogen cyanide represents a significant health and safety risk that must be accounted for prior to and during production of MAA/MMA. Several other petrochemical routes to MAA/MMA exist as well. Renewable routes to MAA and MMA are of interest to obviate the deficiencies of the conventional petrochemical methods while providing a bio-based, drop-in replacement for an otherwise nonrenewable material.

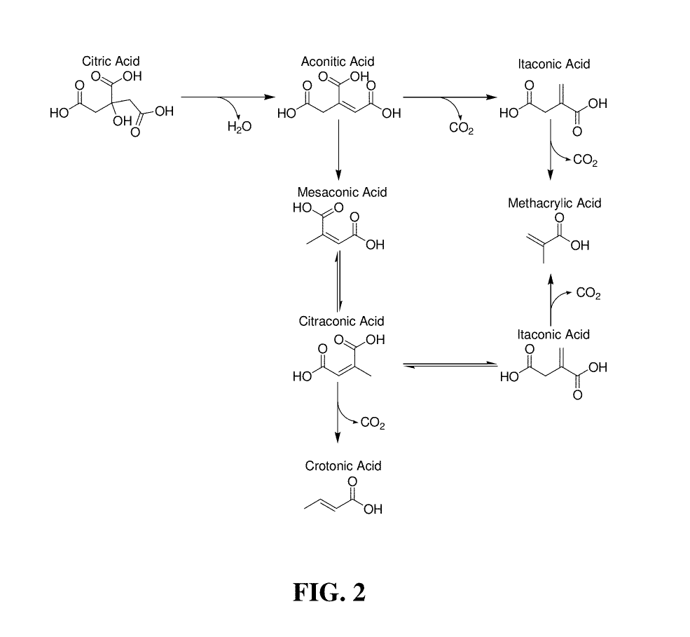

Relatively few bio-based routes to MAA have been reported. One such approach is liquid-phase dehydration and decarboxylation of citramalic (2-hydroxy-2-methylbutanedioc) acid at elevated temperatures (250-400° C.) and pressures (450-5,070 psi) in the presence catalytic sodium hydroxide (U.S. Pat. No. 8,933,179 to Johnson, et al., the entirety of which is incorporated herein by reference). Application of this methodology to maleic (2Z-butenedioc) acid yields acrylic (2-propenoic) acid (AA). Another approach is the Alpha Process, which relies on carbon monoxide, methanol and ethylene to yield MMA following a two-step sequence (WO Publication WO 09619434 by Tooze, et al., the entirety of which is incorporated herein by reference). The first step entails catalytic carbonylation of ethylene in the presence of carbon monoxide to yield methyl propionoate. Subsequent condensation with formaldehyde gives MMA. However, considerable processing and purification of biomass sources is required to obtain the necessary chemical feedstocks (methanol, carbon monoxide and ethylene), which is energy intensive. A further approach involves dehydration and selective decarboxylation of citric acid to provide itaconic (2-methylidene-1,4-butanedioc) acid (IA), followed by a second decarboxylation to yield MAA in the presence of stoichiometric bases under near-critical and supercritical water conditions (FIG. 2, Carlsson, M.; Habenicht, C.; Kam, L. C.; Antal Jr., M. J.; Bian, N.; Cunningham, R. J.; and Jones Jr., M. Study of sequential conversion of citric to itaconic to methacrylic acid in near-critical and supercritical water, Industrial & Engineering Chemistry Research. 33:1989-1996, 1994; the entirety of which is incorporated herein by reference). However, appreciable accumulation of byproducts such as 2-hydroxyisobutyric (HBA) and crotonic [2E-butenoic] acids (CA) at the expense of MAA was problematic. In addition to low selectivity, high temperatures (>350° C.) were needed to achieve relatively low (>70%) yields. Using a supercritical water approach, selectivity was reportedly improved to over 90%, but high temperatures (245-270° C.) and pressures (450-3000 psi) were still needed, stoichiometric amounts of bases were used, and byproducts such as CA and HBA were produced along with propylene (WO patent publication WO 2012/069813 by Johnson, et al., the entirety of which is incorporated herein by reference). Propylene arises via unwanted decarboxylation of MAA and/or CA, thereby reducing the yield of the intended product.

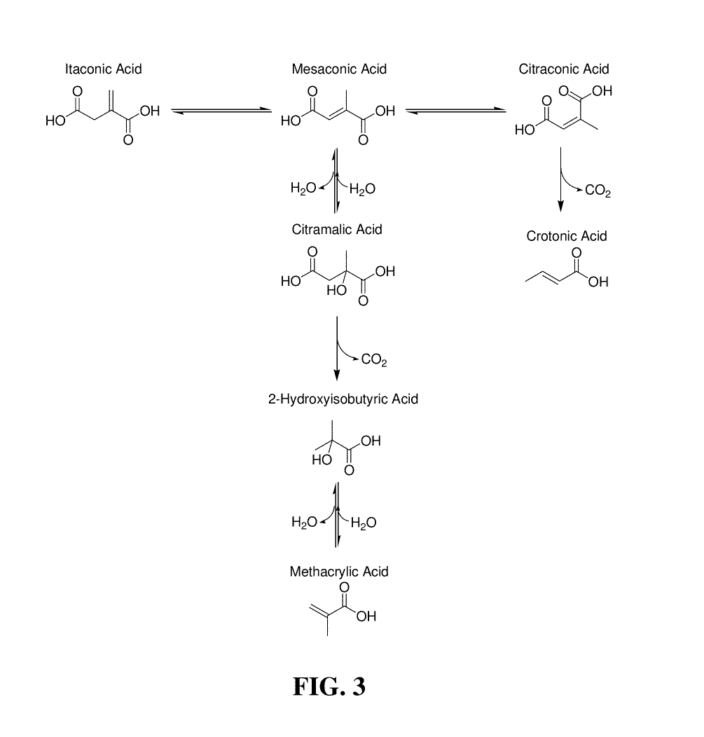

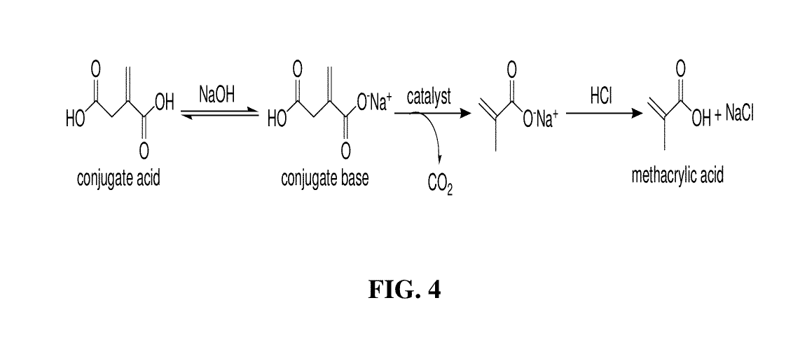

More recently a catalytic approach was described whereby either IA or citric acid was decarboxylated using heterogeneous catalysts based on palladium (Pd/C and Pd/Al₂O₃), platinum (Pt/C and Pt/A₂O₃) and ruthenium (Ru/C) (Le Notre, J.; Witte-van Dijk, C. M.; van Haveren, J.; Scott, E. L.; and Sanders, J. P. M. Synthesis of bio-based methacrylic acid by decarboxylation of itaconic acid and citric acid catalyzed by solid transition-metal catalysts, ChemSusChem 7:2712-2720, 2014; the entirety of which is incorporated herein by reference). Higher selectivity was allegedly achieved (up to 84%) and lower reaction temperatures (200-250° C.) and pressures (550 psi) were utilized relative to previous contributions (FIG. 3). However, similar to previous contributions, stoichiometric bases such as sodium hydroxide (NaOH) were utilized during the reaction. In such embodiments, NaOH reacts with the starting acid to form a monosodium carboxylate salt (conjugate base), which is then decarboxylated to sodium methacrylate. Subsequent acidification of the sodium salt is required to generate MAA, thereby adding basification and acidification steps to the process while consuming additional water that must be removed to isolate the desired product (FIG. 4). In essence this approach converts the conjugate base of IA to the conjugate base of MAA, rather than IA directly to MAA. Other salts (conjugate bases) such as potassium, ammonium and others may also be formed by reaction with the appropriate bases. Consequently, there is a need for a bio-based technology that avoids basification and acidification steps through direct decarboxylation of the starting acid to MAA.

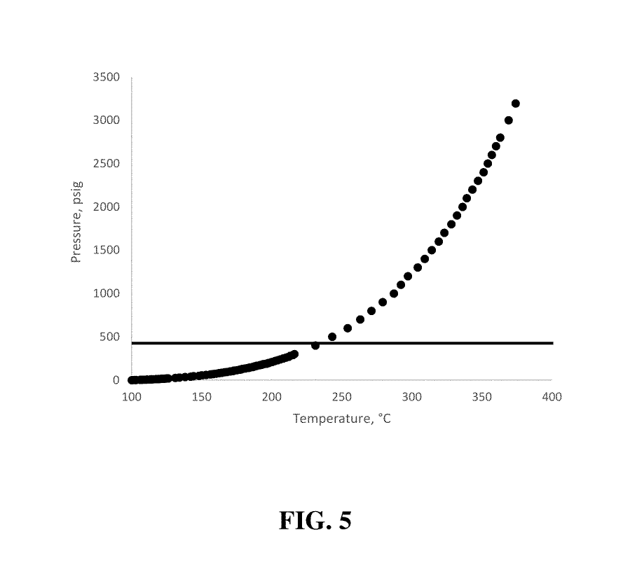

Another disadvantage to prior art is low concentrations of starting material in aqueous solvent. Low concentrations (≤1 M) not only reduce product throughout but also increase energy demands associated with heating reaction mixtures and subsequent solvent removal. Another significant consequence of low starting material concentration is that the majority of the reaction vessel is thus occupied by water, thus increasing pressure due to the very high vapor pressure of water, as seen from the vapor pressure curve of water depicted in FIG. 5. Such conditions therefore lead to supercritical or near supercritical water pressures inside of the reaction vessel. Higher reaction pressures require more expensive reactors that are designed to accommodate such harsh conditions. Consequently, there is a need for a bio-based technology that avoids low substrate concentrations to improve product throughput, has lower energy requirements and reduces reaction pressures.

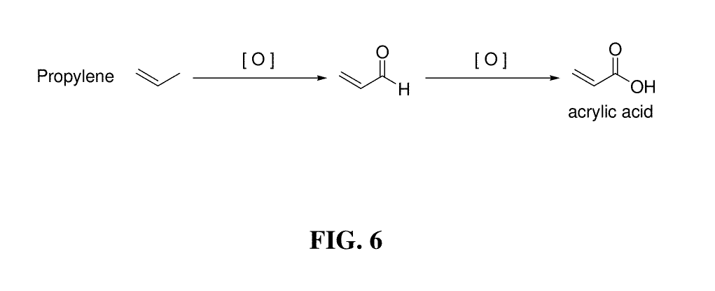

Further, there are disadvantages in the commercial production of acrylic acid (AA). The traditional petrochemical route to AA begins with catalytic oxidation of propylene to yield acrolein followed by a second catalytic oxidation to provide AA (FIG. 6). Other commercial production methods start with acetylene, acrylonitrile, ethylene oxide, ketene, and acetic acid. Similar to petrochemical routes to MAA, none of these technologies utilize renewable materials, and toxic reagents, intermediates, and/or byproducts are produced in many cases. Bio-based routes to AA are therefore of interest to obviate the various deficiencies of the environmentally unfriendly conventional petrochemical methods. Various other (including bio-based) approaches are reported for production of AA. Unfortunately, these approaches have many downsides, such as side reactions leading to acetaldehyde, propionic acid, 2,3-pentanedione, and dilactide result in low yield and selectivity, the high temperatures and pressures necessary, the requirement that expensive catalysts be used, and the difficulty of removal of byproducts (See, for example, U.S. Pat. No. 2,469,701 to Redmon). Lastly, supercritical water approaches similar to the IA→MAA process discussed previously are reported for conversion of fumaric acid (FA) to AA. Disadvantages described previously inherent to the IA to MAA conversion also apply to the reported conversion of FA to AA. Therefore, a commercially feasible, bio-based route to AA or the corresponding alkyl esters that is largely free of these technical difficulties is desired.

All of the references cited herein, including U.S. Patents and U.S. Patent Application Publications, are incorporated by reference in their entirety.

### SUMMARY

A novel method for the catalytic selective decarboxylation of a starting material to produce an organic acid is disclosed. According to at least one embodiment, the method may include placing a reaction mixture into a reaction vessel, the reaction mixture including a solvent, a starting material, and a catalyst, subjecting the reaction mixture to a predetermined pressure and temperature, and allowing the reaction to continue for 1-3 hours. The starting material may be at least one of a dicarboxylic acid, a tricarboxylic acid, and an anhydride of a dicarboxylic or tricarboxylic acid. The reaction mixture may also include a polymerization inhibitor.

Further, the organic acid may be one of acrylic acid or methacrylic acid.

Further, the predetermined temperature may be between 190° C. and 250° C., or may more preferably be between 200° C. and 240° C., or may most preferably be between 210° C. and 225° C.

Further, the predetermined pressure may be greater than atmospheric pressure but lower than 425 psi.

Further, the catalyst may be one of a metal oxide, metal oxalate, metal acetylacetonate, metal carbonate, metal formate, metal 2-ethylhexanoate, metal carbonyl, metal carbonyl carboxylate, and a combination thereof. An exemplary list of catalysts includes manganese (III) oxide [Mn₂O₃], manganese (IV) oxide [MnO₂], iron (III) oxide [Fe₂O₃], cobalt (II) oxide [CoO], nickel (II) oxide [NiO], copper (II) oxide [CuO], zinc (II) oxide [ZnO], ruthenium (IV) oxide [RuO₂], chromium (III) oxide [Cr₂O₃], vanadium (III) oxide [V₂O₃], aluminum (III) oxide [Al₂O₃], zirconium (IV) oxide [ZrO₂], manganese (II) oxalate [Mn(C₂O₄)], iron (II) oxalate [Fe(C₂O₄)], cobalt (II) oxalate [Co(C₂O₄)], nickel (II) oxalate [Ni(C₂O₄)], copper (II) oxalate [Cu(C₂O₄)], zinc (II) oxalate [Zn(C₂O₄)], potassium (I) chromium (III) oxalate [K₃Cr(C₂O₄)₃], manganese (II) acetylacetonate [Mn(C₅H₈O₂)], iron (III) acetylacetonate [Fe₂(C₅H₈O₂)₃], cobalt (II) acetylacetonate [Co(C₅H₈O₂)], nickel (II) acetylacetonate [Ni(C₅H₈O₂)], copper (II) acetylacetonate [Cu(C₅H₈O₂)], zinc (II) acetylacetonate [Zn(C₅H₈O₂)], bis(acetylacetonato) dioxomolybdenum (VI) [MoO₂(C₅H₈O₂)₂], manganese (II) carbonate [MnCO₃], cobalt (II) chloride [CoCl₂], manganese (II) formate [Mn(CHO₂)₂], cobalt (II) 2-ethylhexanoate [Co(C₈H₁₅O₂)₂], manganese (II) 2-ethylhexanoate [Mn(C₈H₁₅O₂)₂], dimanganese (0) decacarbonyl [Mn₂(CO)₁₀], iron (0) pentacarbonyl [Fe(CO)₅], triruthenium (0) dodecacarbonyl [Ru₃(CO)₁₂], an organophosphine of the type PR₃, PR₂H, or PRH₂, such as triphenylphosphine, a ruthenium (I) dicarbonyl carboxylate [Ru(C_{w}HₓO_{y}N_{z})(CO)₂], wherein W, X, Y and Z are each whole numbers or zero, and a combination thereof.

Further, the catalyst may be present in an amount of 0.1-10 mol % relative to the starting material.

Further, the solvent may comprise water. In addition, a co-solvent may be used to decrease the effective vapor pressure and thus lower the pressure generated during the course of the reaction.

Further, the concentration of the starting material may be 1-10 M, or may more preferably be 4-6 M.

Further, the polymerization inhibitor may be one of hydroquinone, phenothiazine, methylene blue, 4-methoxyphenol, and a combination thereof.

Further, after the initial reaction has completed, the produced organic acid may be separated from a residue remaining in the rest of the reaction mixture. The residue may contain unreacted starting material, and thus the unreacted starting material may be re-subjected to the catalytic reaction conditions to produce additional organic acid. The second reaction may be performed in the same or a different reaction vessel as the first reaction.

According to a further embodiment, an organic acid may be produced by combining a starting material with a catalyst to form a reaction mixture, and then exposing the reaction mixture to predetermined reaction conditions, including a predetermined pressure and a predetermined temperature, for a predetermined time. The organic acid may be one of acrylic acid and methacrylic acid, and the starting material may be a bio-based starting material, including at least one of a dicarboxylic acid, a tricarboxylic acid, and an anhydride of a dicarboxylic or tricarboxylic acid.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

Exemplary FIG. 1 shows production of methacrylic acid from acetone by a conventional petrochemical acetone-cyanohydrin route

Exemplary FIG. 2 shows a bio-based route to methacrylic acid from citric acid via an itaconic acid intermediate.

Exemplary FIG. 3 shows a bio-based route to methacrylic acid directly from itaconic acid with several intermediates.

Exemplary FIG. 4 shows production of methacrylic acid through decarboxylation of monosodium itaconate, the conjugate base of itaconic acid.

Exemplary FIG. 5 shows vapor pressure (psig) of steam as a function of temperature (Celsius). The horizontal line depicted below represents a vapor pressure of 425 psig, which is the maximum preferred partial water vapor pressure according to at least one embodiment. Data presented was obtained from Perry's Chemical Engineers' Handbook (Perry, R. H., Green, D. W., 7^{th} Ed, McGraw-Hill, New York, 1997).

Exemplary FIG. 6 shows a conventional petrochemical production of acrylic acid by oxidation of propylene via a toxic acrolein intermediate.

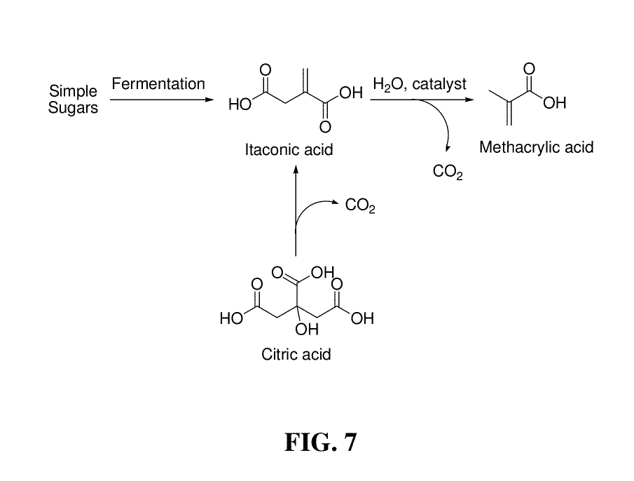

Exemplary FIG. 7 shows bio-based production of methacrylic acid from simple sugars and citric acid via an itaconic acid intermediate.

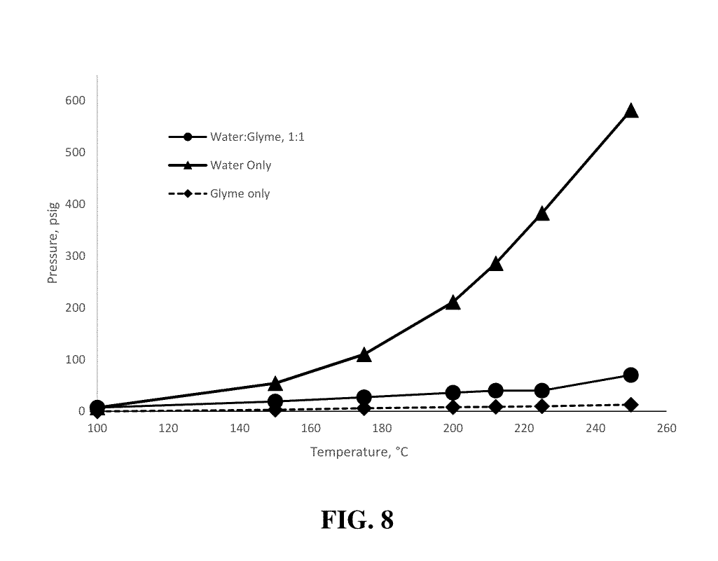

Exemplary FIG. 8 shows a comparison of pressure (psig) as a function of temperature (Celsius) at constant volume among pure water, a 1:1 (mole ratio) mixture of water and tetraglyme (labelled “glyme”), and pure tetraglyme.

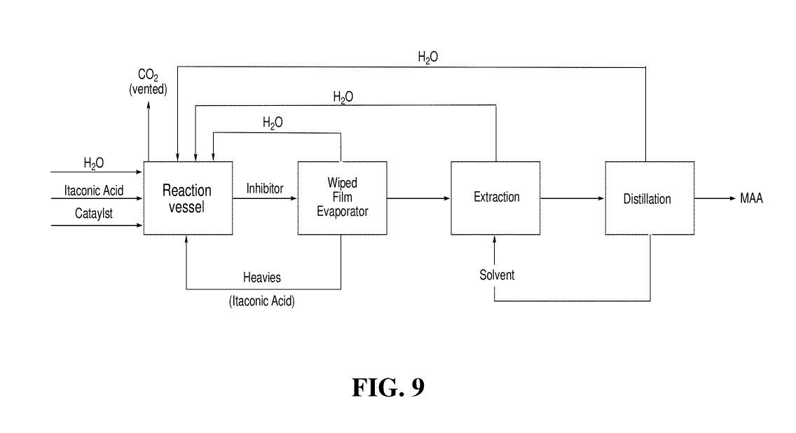

Exemplary FIG. 9 shows a scheme for industrial production of methacrylic acid from itaconic acid according to the present invention, which could be utilized in either a batch or a continuous production process.

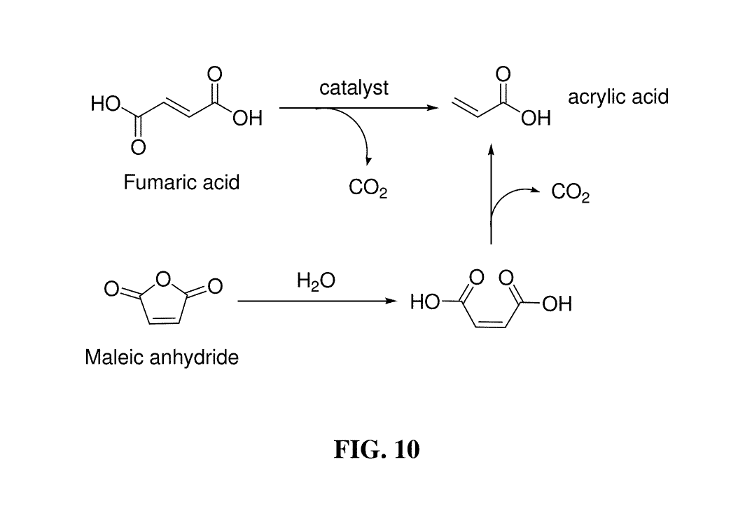

Exemplary FIG. 10 shows two alternative bio-based production routes for acrylic acid, one from fumaric acid and the other from maleic anhydride.

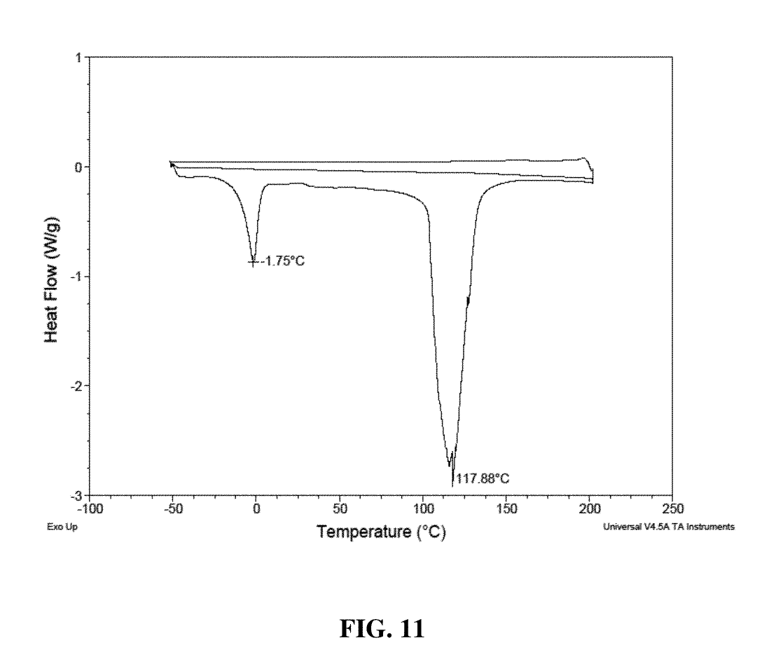

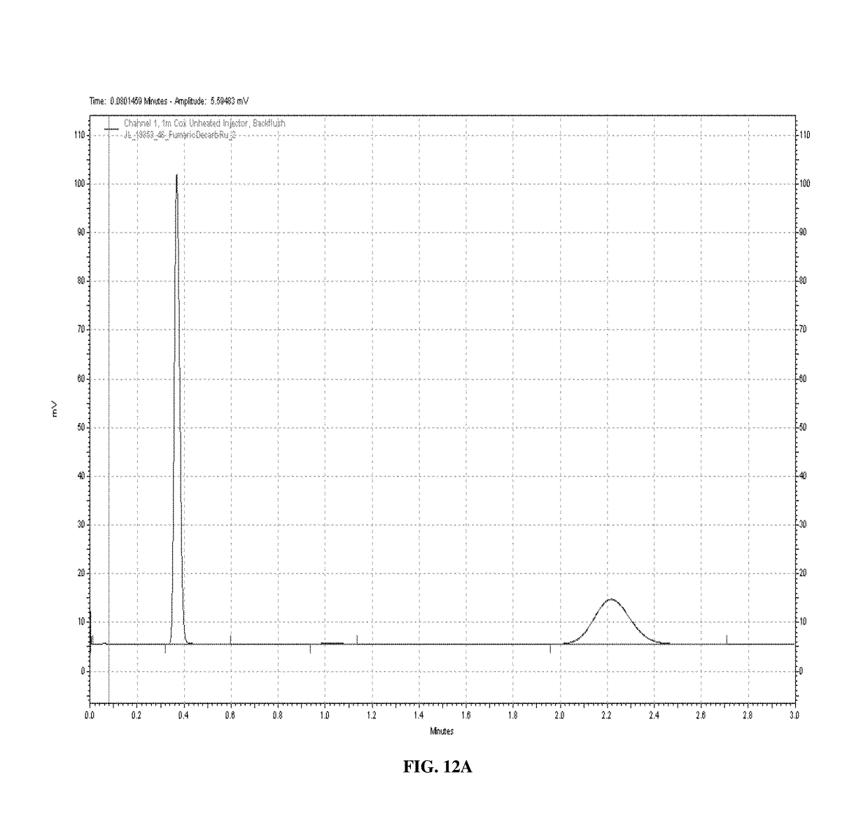

Exemplary FIG. 11 shows differential scanning calorimetry of solid particulate formed as the result of the decarboxylation of fumaric acid with a catalytic amount of [Ru(CH₃CH₂COO)(CO)₂]ₙ.

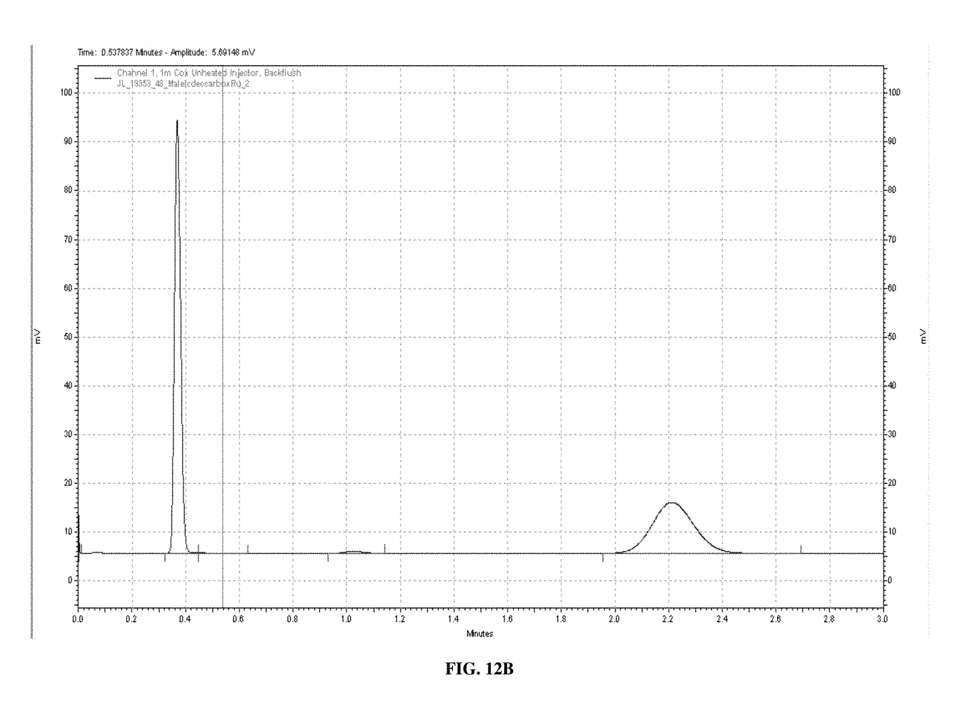

Exemplary FIG. 12A shows a gas chromatograph of the resulting headspace gas after decarboxylation of fumaric acid with a catalytic amount of [Ru(CH₃CH₂COO)(CO)₂]ₙ.

Exemplary FIG. 12B shows a gas chromatograph of the resulting headspace gas after decarboxylation of maleic anhydride acid with a catalytic amount of [Ru(CH₃CH₂COO)(CO)₂]ₙ.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

Unless defined otherwise, all technical and scientific terms used herein have the same meaning as commonly understood by one of ordinary skill in the art to which the invention belongs. As used herein, the term “about” refers to a quantity, level, value, or amount that varies by as much as 30%, preferably by as much as 20%, and more preferably by as much as 10% to a reference quantity, level, value, or amount. Although any methods and materials similar or equivalent to those described herein can be used in the practice or testing of the present invention, the preferred methods and materials are now described.

Further, other compounds may be added to the reaction mixture (such as, e.g. triphenylphosphine) provided they do not substantially interfere with the intended activity and efficacy of the reaction medium; whether or not a compound interferes with activity and/or efficacy can be determined, for example, by the procedures utilized below.

Unless otherwise stated, the amounts, percentages, and ranges disclosed herein are not meant to be limiting, and increments between the recited amounts, percentages, and ranges are specifically envisioned as part of the invention.

The invention described herein achieves direct catalytic decarboxylation of a starter material to achieve the final product. For example, direct catalytic decarboxylation of IA to MAA can be achieved without the need for stoichiometric treatment of starting acid with base nor subsequent acidification to afford MAA. IA may be a preferable starting material for obtaining MAA since it is derived from dehydration and decarboxylation of citric acid or from fermentation of simple disaccharide sugars such as glucose. Other starting materials may include citraconic acid (CCA), mesaconic acid (MSA), tricarboxylic acid, citric acid, or a mixture of acids. The concentration of the starting material in the reaction medium may be 1-10 M, more preferably 4-6 M, and most preferably about 5 M. A diagram showing an exemplary embodiment of the invention is shown in FIG. 7.

Another distinguishing feature of the present invention is the employment of high concentrations of starting material in solvent, thereby enhancing throughput while simultaneously reducing costs associated with heating and cooling of large volumes of water or other solvent. From a chemical standpoint, a near stoichiometric amount of water may be beneficial to mitigate formation of a less chemically reactive anhydride from the starting acid. However, no additional benefit is gained from higher amounts of water. Because the present approach is truly catalytic, it is possible to achieve decarboxylation at lower temperatures than in previously reported methods. Lower temperatures are advantageous because the energy demands of heating the system are lower and because lower temperatures give lower pressures (at constant volume) at reaction temperatures, according to Gay-Lussac's Law. Lower pressures are desirable from engineering and economic standpoints because they are safer and because less expensive reactor designs can be utilized. For example, decarboxylation according to the present invention may be conducted in a temperature range of 190 to 250° C., and more preferably 200 to 240° C., and most preferably 210 to 225° C. When maintaining the temperature inside a reaction vessel, it is understood that some variation in temperature may occur as the reaction progresses, as would be understood by one in the art. Further, decarboxylation according to the present invention may be conducted with an initial headspace pressure of less than 500 psi (approximately 485 psig), and more preferably at 20 to 425 psi (approximately 5 to 410 psig). At such reaction temperatures and pressures, the reaction medium may be in a liquid phase. Further, the reactants and catalyst may be in an aqueous solution or aqueous co-solvent solution. Due to the nature of the starting materials, the pH of the reaction medium may be acidic.

A further aspect of the present invention is the employment of polymerization inhibitors (PI) to prevent loss of MAA to polymerization. MAA is highly polymerizable, which, when used in an industrial scale, must be inhibited. For example, 4-methoxyphenol (MEHQ) may be used as a PI. Other PIs may include hydroquinone, phenothiazine, or methylene blue. The PI may be added to the reaction mixture in a concentration range of 1000-30,000 ppm.

Further, another aspect of the present invention is the use of co-solvents to help with dissolution of all of the reagents and/or lower vapor pressure. Any suitable co-solvent can be used. For the purposes of example, high-boiling organic co-solvents miscible with water in all proportions that are compatible with starting material(s), catalyst(s), and PI(s), such as tetraglyme, are reported herein to simultaneously solubilize the PI and to further lower vapor pressure. The organic solvent may additionally include glyme (dimethoxyethane), diglyme, triglyme, or any capped organic ether. The solvent may be added to the reaction mixture in a range of 0-100 vol % with respect to water. As seen in FIG. 8, lower pressures are achieved in water/tetraglyme mixtures as opposed to in solely aqueous systems. In combination, the innovations described herein represent techno-economic improvements over existing methodologies, thus yielding a bio-based route to an important industrial monomer.

The present invention utilizes a catalyst. The catalyst may be homogenous or heterogeneous. Further, the catalyst may be a metal oxide, oxalate, acetylacetonate, carbonate, chloride, formate, 2-ethylhexanoate, carbonyl, or carbonyl carboxylate. A non-limiting list of potential catalysts includes manganese (III) oxide [Mn₂O₃], manganese (IV) oxide [MnO₂], iron (III) oxide [Fe₂O₃], cobalt (II) oxide [CoO], nickel (II) oxide [NiO], copper (II) oxide [CuO], zinc (II) oxide [ZnO], ruthenium (IV) oxide [RuO₂], chromium (III) oxide [Cr₂O₃], vanadium (III) oxide [V₂O₃], aluminum (III) oxide [Al₂O₃], zirconium (IV) oxide [ZrO₂], manganese (II) oxalate [Mn(C₂O₄)], iron (II) oxalate [Fe(C₂O₄)], cobalt (II) oxalate [Co(C₂O₄)], nickel (II) oxalate [Ni(C₂O₄)], copper (II) oxalate [Cu(C₂O₄)], zinc (II) oxalate [Zn(C₂O₄)], potassium (I) chromium (III) oxalate [K₃Cr(C₂O₄)₃], manganese (II) acetylacetonate [Mn(C₅H₈O₂)], iron (III) acetylacetonate [Fe₂(C₅H₈O₂)₃], cobalt (II) acetylacetonate [Co(C₅H₈O₂)], nickel (II) acetylacetonate [Ni(C₅H₈O₂)], copper (II) acetylacetonate [Cu(C₅H₈O₂)], zinc (II) acetylacetonate [Zn(C₅H₈O₂)], bis(acetylacetonato) dioxomolybdenum (VI) [MoO₂(C₅H₈O₂)₂], manganese (II) carbonate [MnCO₃], cobalt (II) chloride [CoCl₂], manganese (II) formate [Mn(CHO₂)₂], cobalt (II) 2-ethylhexanoate [Co(C₈H₁₅O₂)₂], manganese (II) 2-ethylhexanoate [Mn(C₈H₁₅O₂)₂], dimanganese (0) decacarbonyl [Mn₂(CO)₁₀], iron (0) pentacarbonyl [Fe(CO)₅], triruthenium (0) dodecacarbonyl [Ru₃(CO)₁₂], organophosphines of the type PR₃, PR₂H, or PRH₂, such as triphenylphosphine, and ruthenium (I) dicarbonyl carboxylates [Ru(C_{w}HₓO_{y}N_{z})(CO)₂] where W, X, Y and Z are whole numbers or zero, with a representative example being poly(ruthenium (I) propionato-dicarbonyl) [Ru(C₃H₅O₂)(CO)₂]ₙ.

The catalyst may be present in the amount of 0.1-10.0 mol % relative to the amount of starting material. The mole percent of a catalyst containing ruthenium may preferably be 0.1-0.5 mol % relative to the amount of the starting material, and may be most preferably about 0.1 mol %. The mole percent of a non-ruthenium catalyst may preferably be 1.0-10.0 mol % relative to the amount of the starting material, more preferably 2.5-5.0 mol %, and most preferably about 5.0 mol %

According to some embodiments, the catalyst used may preferably be manganese (II) oxalate or ruthenium (I) carbonyl carboxylate. These are hitherto unreported as catalysts for selective decarboxylation of IA. Heterogeneous ruthenium on carbon (Ru/C) was described in prior art for such a transformation, but was reported to be less desirable than platinum on aluminum oxide (Pt/Al₂O₃) (Le Notre, et al.). Ruthenium (I) carbonyl carboxylates such as ruthenium (I) propionate dicarbonyl and triruthenium (0) dodecacarbonyl have been described as catalyst precursors for tandem isomerization and decarboxylation of unsaturated fatty acids to a mixture heptadecene isomers (see, for example, Pat. App. Pub. No. 20140275592 by Murray, et al., the entirety of which is incorporated herein by reference), but dioic or polyoic acids such as IA or citric acid have not been investigated before now.

The method of the present invention may be utilized in either a batch mode or a continuous mode. The examples and procedures described herein represent batch mode decarboxylations conducted without periodic venting of the headspace above the reaction mixture inside of the enclosed vessel. The headspace was not vented due to a desire to characterize the gases produced during the course of the reaction. As a result, final pressures in the examples described herein were higher than they would have been had the headspace been vented. However, a commercial process utilizing the present invention may preferably periodically or continuously vent the carbon dioxide produced during the course of the reaction to give lower vessel pressures. Furthermore, a preferred commercial embodiment of the invention may not only include pressure venting but would also be conducted in continuous mode with recycling of unreacted starting materials, catalysts and solvents to give additional MAA, as depicted in FIG. 9.

Separation of MAA from the starting material, catalyst(s), and oligomers/polymers (if produced) may be accomplished using a commercial thin or wiped film evaporator, and/or via azeotropic distillation, for example. The heavier components from the separation (such as, for example, starting materials, catalyst(s), and oligomers/polymers) may then be recycled back to the reactor for further reaction to give additional MAA. The light fraction exiting the wiped film evaporator may consist of MAA and water, which may then enter an industrial-scale extractor for separation of water from MAA. The organic extraction solvent may then be removed and recycled back into the extractor via distillation to provide high-purity MAA stabilized with a PI. Water removed during the course of decantation and distillation may be recycled back into the reaction vessel. MAA can then be derivatized to any number of commercially significant esters and/or polymers.

The present invention may also be useful for the conversion of fumaric acid (FA; but-2E-enedioc acid), maleic acid (MA; but-2Z-enedioc acid) or maleic anhydride (MAN; 2,5-furandione) into AA, as depicted in FIG. 10.

Thus, since the chemical formula of acrylic acid is CH₂CHCOOH and methacrylic acid is CH₂CCH₃COOH, the present invention may be described as a method for producing an organic acid having the chemical formula of CH₂CRCOOH, where R is either H or CH₃.

### EXAMPLE 1

### Production of MAA with Various Catalysts

Methacrylic acid was produced via decarboxylation according to the following method and using the following materials. Water for decarboxylation reactions and as a component of the mobile phase for HPLC was ultra-pure (18 MSΩ) obtained from a Barnstead (Lake Balboa, Calif.) Easy Pure II RF/UV ultrapure water system. Prior to decarboxylation, ultra-pure water was degassed by passing nitrogen through it for several hours. Aluminum MD oxide (Al₂O₃; 99.9%), cobalt (II) acetylacetonate [Co(acac)₂; 99%], crotonic acid [2E-butenoic acid; 98%), iron (III) acetylacetonate [Fe(acac)₃; 99.9%], iron (II) oxalate monohydrate (FeC₂O₄·H₂O; 96%), itaconic acid (2-methylidenebutanedioc acid; 99%), manganese (II) acetylacetonate [Mn(acac)₂; 99%], manganese (II) oxalate dihydrate (MnC₂O₄·2H₂O; 99%), manganese (I) oxide (Mn₂O; 99.9%), nickel (II) acetylacetonate [Ni(acac)₂; 95%], zinc (II) acetylacetonate monohydrate [Zn(acac)₂·H₂O; 98%], and zinc (II) oxide (ZnO; 99%) were purchased from Alfa Aesar (Ward Hill, Mass.). Acrylic acid (2-propenoic acid; 99%), citraconic acid [(2Z)-2-methylbut-2-enedioc acid; 98%], cobalt (II) oxalate dihydrate (CoC₂O₄·2H₂O; 99%), cobalt (II) oxide (CoO; 99%), chromium (III) oxide (Cr₂O₃; 99.9%), copper (II) oxide (CuO; 99.9%), iron (III) oxide (Fe₂O₃; 99.9%), methacrylic acid (2-methylpropenoic acid; 99%), mesaconic acid [(2E)-2-methylbut-2-enedioc acid; 98%], nickel (II) oxide (NiO; 99.9%), potassium chromium oxalate trihydrate [K₃Cr(C₂O₄)₃·3H₂O; 99%], maleic anhydride (2,5-furandione; 99%), 4-methoxyphenol (MEHQ; 99%), triphenylphosphine (PPh₃; 99%), and trifluoroacetic acid (CF₃CO₂H, TFA, 99%) were obtained from Sigma-Aldrich Corp (St. Louis, Mo.). Copper (II) acetylacetonate [Cu(acac)₂; 98%] and fumaric acid [(2E)-but-2-enedioic acid; 99+%] were purchased from Acros Organics (Morris Plains, N.J.). Propionato-dicarbonyl ruthenium (I) was prepared according to literature precedent (Crooks, et al. Chemistry of polynuclear compounds Part XVII; Some carboxylate complexes of ruthenium and osmium carbonyls, J. Chem. Soc. A. 2761-2766; 1969). All other materials were obtained from Sigma-Aldrich Corp and used as received. Catalysts, IA, and FA were stored prior to use in a nitrogen filled Innovative Technologies (Amesbury, Mass.) model IL-2GB inert atmosphere glove-box with atmospheric oxygen and water concentrations kept below 1 ppm.

Decarboxylations were performed using a Parr Instrument Company (Moline, Ill.) 50 mL stainless steel (T316) reactor with a maximum allowable working pressure of 3,000 psi at 350° C. The reactor was lined with a glass liner and equipped with an internal thermocouple, cooling loop, variable speed overhead magnetic stirrer with a standard shaft and impeller, heater assembly, and a Span (Waukesha, Wis.) overhead pressure gauge rated up to 3,000 psi. Internal reaction temperature and stirring rate (rpm) were controlled by a Parr model 4848 reactor controller unit, which also provided internal vessel pressure information.

For each decarboxylation, IA (1.0 M in water) and catalyst (10%) were weighed in the inert atmosphere glove box and placed in a glass liner. A rubber septum was affixed to the glass liner, which was then removed from the glove box. Ultra-pure and degassed water (14.0 mL) was added to the closed system using a syringe, and the septum was removed before the liner was placed in the reaction vessel. After the reactor was closed and purged three times with nitrogen, the headspace of the vessel was pressurized to 100 psi (at room temperature) using nitrogen. The mixture was then stirred (500 rpm) and heated over the course of approximately 20 min to the reaction temperature (250° C.). The temperature was then maintained while the mixture was allowed to react for 3.0 hr; the recorded reaction time started when the desired temperature was reached. After the allocated reaction time, the reactor was equilibrated to room temperature (1-1.5 hr) and the gas pressure was released. The crude solution was purified by extraction utilizing ethyl acetate or by azeotropic distillation of the aqueous solution. For distillation, the aqueous mixture was transferred to a 2-neck flask affixed with a short path distillation head leading to a 4 flask receiver. A clear solution was distilled over at 100° C. and collected. The initial solution, as well as the distillate and the residue, were analyzed by High Performance Liquid Chromatography (HPLC). The results are shown in Table 1 below:

TABLE 1

| | Time | Temp | | | | | p_{H2O}³ |
|---|---|---|---|---|---|---|---|
| Catalyst | (h) | (° C.) | % IA¹ | % MAA¹ | % CA¹ | Selectivity² | (psig) |
| Control (no catalyst) | 3.0 | 250 | 52.5 | 8.8 | 0.2 | 97.8 | 540 |
| Mn (II) oxalate | 3.0 | 250 | 8.6 | 35.6 | 2.0 | 94.8 | 540 |
| Fe (II) oxalate | 3.0 | 250 | 11.9 | 31.1 | 4.6 | 87.1 | 540 |
| Co (II) oxalate | 3.0 | 250 | 12.1 | 33.3 | 4.4 | 88.2 | 540 |
| Cu (II) oxalate | 3.0 | 250 | 12.6 | 25.9 | 10.6 | 71.0 | 540 |
| Zn (II) oxalate | 3.0 | 250 | 11.1 | 30.3 | 4.3 | 87.7 | 540 |
| MoO₂(acac)₂ | 3.0 | 250 | 44.5 | 11.9 | 1.2 | 91.0 | 540 |
| Mn (II) acac | 3.0 | 250 | 7.2 | 21.5 | 20.5 | 51.2 | 540 |
| Fe (III) acac | 3.0 | 250 | 12.6 | 21.4 | 15.3 | 58.3 | 540 |
| Co (II) acac | 3.0 | 250 | 6.2 | 11.6 | 33.4 | 25.8 | 540 |
| Cu (II) acac | 3.0 | 250 | 29.3 | 16.9 | 6.9 | 71.2 | 540 |
| Zn (II) acac | 3.0 | 250 | 8.8 | 23.3 | 20.3 | 53.5 | 540 |
| Ni (II) acac | 3.0 | 250 | 5.5 | 10.6 | 39.4 | 21.3 | 540 |
| Mn (III) oxide | 3.0 | 250 | 11.6 | 34.8 | 3.6 | 90.6 | 540 |
| Mn (IV) oxide | 3.0 | 250 | 12.0 | 32.6 | 3.5 | 90.3 | 540 |
| Fe (III) oxide | 3.0 | 250 | 38.4 | 13.7 | 1.1 | 92.4 | 540 |
| Co (II) oxide | 3.0 | 250 | 11.6 | 32.0 | 4.3 | 88.2 | 540 |
| Cu (II) oxide | 3.0 | 250 | 17.5 | 21.4 | 8.8 | 70.9 | 540 |
| Zn (II) oxide | 3.0 | 250 | 11.5 | 31.2 | 5.1 | 86.0 | 540 |
| Ni (II) oxide | 3.0 | 250 | 24.4 | 24.6 | 2.2 | 91.8 | 540 |
| Cr (III) oxide | 3.0 | 250 | 43.0 | 13.6 | 0.9 | 94.0 | 540 |
| V (III) oxide | 3.0 | 250 | 45.5 | 12.5 | 0.5 | 96.2 | 540 |
| Ru (IV) oxide | 3.0 | 250 | 28.9 | 10.1 | 0 | 100 | 540 |
| Al (III) oxide | 3.0 | 250 | 42.7 | 13.2 | 0.8 | 94.5 | 540 |
| Zr (IV) oxide | 3.0 | 250 | 37.2 | 14.4 | 0.8 | 95.0 | 540 |
| Mn(II)CO₃ | 0.5 | 170 | 60.9 | 14.0 | 0.2 | 98.6 | 98 |
| Co(II)Cl₂ | 2.0 | 240 | 65.1 | 9.3 | 0.5 | 94.9 | 235 |
| Mn(II)[(CHO₂)₂) | 2.0 | 240 | 19.8 | 28.7 | 6.3 | 82.0 | 235 |
| Co(C₈H₁₅O₂)₂ | 2.0 | 240 | 51.0 | 15.3 | 2.6 | 85.5 | 235 |
| Mn(C₈H₁₅O₂)₂ | 2.0 | 240 | 59.8 | 15.7 | 2.1 | 88.2 | 235 |
| Mn₂(CO)₁₀ | 1.5 | 240 | 7.9 | 5.3 | 0.4 | 93.0 | 235 |
| Ru₃(CO)₁₂ | 2.0 | 240 | 1.5 | 7.9 | 0 | 100 | 235 |

Results of catalytic decarboxylation of IA with different catalysts.
¹% IA, % MAA, and % CA are overall percentage yields.
²Selectivity is [MAA/(MAA + CA) × 100].
³Partial vapor pressure of water (p_{H2O}, psig) was calculated from mole fraction of water and vapor pressure data from Perry's Chemical Engineers' Handbook, 7^{th} Ed (Perry, R. H.; Green, D. W. McGraw-Hill, New York, 1997)

As seen in Table 1, most of the catalysts examined provided yields of MAA greater than the control. Further, Ru₃(CO)₁₂, while providing slightly less MAA than the control, had 100% selectivity. Further, some catalysts, such as manganese (II) oxalate, manganese (III) oxide, manganese (IV) oxide, and ruthenium (IV) oxide provided improved yields with high selectivities of 90-100%.

### EXAMPLE 2

### Production of MAA with Mn (II) Oxalate, Triphenylphosphine, and PI

Methacrylic acid was produced via decarboxylation according to the same method and using the same materials as in Example 1, except that IA was used in a concentration of 5.5 M, either or both of Mn (II) oxalate and triphenylphosphine (TPP) were used as the catalyst, at a concentration of 5.0 mol % catalyst relative to the starting material, 5,000 ppm of MEHQ was added as a PI, the initial N₂ headspace pressure was 400±5 psig, and the reaction time and temperature was varied to determine the optimal conditions. The results are shown in Table 2 below:

TABLE 2

| Time | Temp | Mn (II) | | | | % other | | p_{H2O}³ |
|---|---|---|---|---|---|---|---|---|
| (h) | (° C.) | oxalate | TPP | % CA | % MAA | products¹ | Selectivity² | (psig) |
| 1.5 | 225 | 5.0 | 0 | 0.8 | 2.6 | 96.6 | 76.5 | 312 |
| 2.0 | 200 | 5.0 | 0 | 0.2 | 7.2 | 92.6 | 97.3 | 191 |
| 1.5 | 200 | 5.0 | 0 | 0.8 | 1.5 | 97.7 | 65.2 | 191 |
| 1.5 | 212 | 5.0 | 0 | 0.8 | 2.4 | 96.8 | 75.0 | 244 |
| 1.5 | 225 | 5.0 | 0 | 0.7 | 4.8 | 94.5 | 87.2 | 312 |
| 1.5 | 225 | 5.0 | 0 | 0.3 | 7.0 | 92.7 | 95.9 | 312 |
| 1.5 | 250 | 5.0 | 0 | 0.9 | 5.7 | 93.4 | 86.4 | 499 |
| 1.5 | 212 | 5.0 | 5.0 | 2.1 | 5.3 | 92.6 | 71.6 | 243 |
| 1.5 | 225 | 5.0 | 5.0 | 2.4 | 13.0 | 84.6 | 84.4 | 311 |
| 1.5 | 225 | 0 | 5.0 | 2.9 | 14.0 | 83.1 | 82.8 | 311 |

Results of catalytic decarboxylation of IA with Mn (II) oxalate and triphenylphosphine.
¹Refers to the sum of other byproducts of the reaction, including IA, CCA, MSA, and others.
²Selectivity was calculated according to the same method as in Table 1.
³Partial vapor pressure of water was calculated according to the same method in Table 1.

As seen in Table 2, the reaction performs well in different conditions, and one preferred embodiment may be conducting the reaction at 225° C. for 1.5 hours. Further, the addition of TPP considerably increased the yield of MAA, from 7.0% to 13.0% (at 225° C.).

### EXAMPLE 3

### Production of MAA with Ruthenium Dicarbonyl Propionate and PI

Methacrylic acid was produced via decarboxylation according to the same method and using the same materials as in Example 1, except that IA was used in a concentration of 5.5 M, only ruthenium dicarbonyl propionate [(Ru(CO)₂(CH₃CH₂COO)]ₙ was used as the catalyst, at a concentration of 0.1 mol % Ru, 5,000 ppm of MEHQ was added as a PI, the reactions were carried out at 400±5 psig using a N₂ headspace, and the reaction time and temperature was varied to determine the optimal conditions. In addition, in some reactions, 0.5 mol % TPP was also added to the reaction mixture to potentially assist with MAA production. The results are shown in Table 3 below:

As seen in Table 3, the addition of TPP considerably increased the yield of MAA, from 8.6% up to at least 33.8%. It's further noted that at 33.8% yield, the selectivity was 100%.

In addition, further experiments were performed demonstrating the use of an organic solvent to lower the reaction pressure. Reactions were performed as in the reactions in Table 3, for 1.5 hours at 225° C. Tetraglyme (TG) was added to demonstrate the effect, and the change in pressure over the course of the reaction (AP) was monitored to quantify results. The results are shown in Table 4 below:

TABLE 4

| mole ratio | mole ratio | | | | % other | | p_{H2O}³ |
|---|---|---|---|---|---|---|---|
| TG:water | IA:water | ΔP psi | % CA | % MAA | products¹ | Selectivity² | (psig) |
| 0:1 | 1:10 | 277 | 0.9 | 14.9 | 84.2 | 94.3 | 313 |
| 1:0 | N/A | 108 | 3.7 | 3.9 | 92.4 | 51.3 | 0 |
| 1:1 | 0.72:1 | 133 | 3.0 | 12.6 | 84.4 | 80.8 | 101 |
| 1:3 | 1:2 | 139 | 3.8 | 7.6 | 88.6 | 66.7 | 188 |
| 1:4 | 1:2.5 | 153 | 3.3 | 9.2 | 87.5 | 73.6 | 209 |

Results of catalytic decarboxylation of IA with [Ru(CO)₂(CH₃CH₂COO)]ₙ with varied solvent compositions.
¹Refers to the sum of other byproducts of the reaction, including IA, CCA, MSA, and others.
²Selectivity was calculated according to the same method as in Table 1.
³Partial vapor pressure of water was calculated according to the same method in Table 1.

As can be seen in Table 4, the addition of an additional solvent such as an organic solvent can significantly reduce the effective pressure of the reaction conditions. However, the low yield of MAA when water is completely replaced by an organic solvent suggests the formation of anhydrides. Thus, according to at least one embodiment, water is a necessary component in the reaction medium to mitigate formation of anhydrides in situ.

### EXAMPLE 4

### Production of MAA with Ruthenium Dicarbonyl Propionate and Citric Acid

Methacrylic acid was produced via decarboxylation according to the same method and using the same materials as in Example 3, except that citric acid was used in a concentration of 5.5 M. As in Example 3, only ruthenium dicarbonyl propionate [(Ru(CO)₂(CH₃CH₂COO)]ₙ was used as the catalyst, at a concentration of 0.1 mol % Ru, 5,000 ppm of MEHQ was added as a PI, the reactions were carried out at 400±5 psig using a N₂ headspace, and the reaction time and temperature was varied to determine the optimal conditions. In addition, as in Example 3, in some reactions, 0.5 mol % TPP was also added to the reaction mixture to potentially assist with MAA production. The results are shown in Table 5 below:

As seen in Table 5, reaction parameters were found such that using citric acid, an MAA yield of at least 24.1% was obtained. It's noted that with the high yield, the selectivity in that experiment was 100%.

### EXAMPLE 5

### Production of AA with Ruthenium Dicarbonyl Propionate

Acrylic acid was produced via decarboxylation according to the same method and using the same materials as in Example 3, except that FA and MAN were used as the starting material, 0.1 mol % [Ru(CH₃CH₂COO)(CO)₂]ₙ was used as the catalyst, the reaction pressure was 400 psig, the reaction temperature was 225° C., and the reaction time was 2 hr. The product was identified as polyacrylic acid by determination of glass transition temperature by differential scanning calorimetry (DSC), the results of which are shown in FIG. 11. The DSC results show an event at −1.75° C., corresponding to water, and an event at 118° C., corresponding to polyacrylic acid (literature value is 106° C.). Analysis of the resulting headspace gas by gas chromatography (GC) confirmed the presence of carbon dioxide, thereby suggesting decarboxylation of the substrate had occurred. GC results are shown in FIG. 12A (for FA) and in FIG. 12B (for MAN). In both of FIGS. 12A and 12B, the peak at 0.4 min corresponds to nitrogen, while the peak at 2.2 min corresponds to CO₂.

### CONCLUSION

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1: A method for the selective production of an organic acid, comprising:
placing a reaction mixture into a first reaction vessel, the reaction mixture comprising a first solvent, a starting material, and a catalyst;
pressurizing the first reaction vessel to a predetermined pressure;
heating the reaction mixture to a predetermined temperature; and
maintaining the predetermined temperature for a predetermined reaction time,
wherein the starting material is a at least one of a dicarboxylic acid, a tricarboxylic acid, and an anhydride of a dicarboxylic or tricarboxylic acid.

2: The method of claim 1, wherein the organic acid has a chemical formula of CH₂CRCOOH, wherein R is either an H or a CH₃.

3: The method of claim 1,
wherein the organic acid is methacrylic acid, and
wherein the starting material is one of itaconic acid, citraconic acid, mesaconic acid, citric acid, and a combination thereof.

4: The method of claim 1,
wherein the organic acid is acrylic acid, and
wherein the starting material is one of fumaric acid, maleic acid, maleic anhydride, and a combination thereof.

5: The method of claim 1, wherein the predetermined temperature is between 190° C. and 250° C.

6: The method of claim 1, wherein the predetermined pressure is between 5-410 psig.

7: The method of claim 1, wherein the catalyst one of a metal oxide, metal oxalate, metal acetylacetonate, metal carbonate, metal formate, metal 2-ethylhexanoate, metal carbonyl, metal carbonyl carboxylate, and a combination thereof.

8: The method of claim 7, wherein the catalyst is one of manganese (III) oxide [Mn₂O₃], manganese (IV) oxide [MnO₂], iron (III) oxide [Fe₂O₃], cobalt (II) oxide [CoO], nickel (II) oxide [NiO], copper (II) oxide [CuO], zinc (II) oxide [ZnO], ruthenium (IV) oxide [RuO₂], chromium (III) oxide [Cr₂O₃], vanadium (III) oxide [V₂O₃], aluminum (III) oxide [Al₂O₃], zirconium (IV) oxide [ZrO₂], manganese (II) oxalate [Mn(C₂O₄)], iron (II) oxalate [Fe(C₂O₄)], cobalt (II) oxalate [Co(C₂O₄)], nickel (II) oxalate [Ni(C₂O₄)], copper (II) oxalate [Cu(C₂O₄)], zinc (II) oxalate [Zn(C₂O₄)], potassium (I) chromium (III) oxalate [K₃Cr(C₂O₄)₃], manganese (II) acetylacetonate [Mn(C₅H₈O₂)], iron (III) acetylacetonate [Fe₂(C₅H₈O₂)₃], cobalt (II) acetylacetonate [Co(C₅H₈O₂)], nickel (II) acetylacetonate [Ni(C₅H₈O₂)], copper (II) acetylacetonate [Cu(C₅H₈O₂)], zinc (II) acetylacetonate [Zn(C₅H₈O₂)], bis(acetylacetonato) dioxomolybdenum (VI) [MoO₂(C₅H₈O₂)₂], manganese (II) carbonate [MnCO₃], cobalt (II) chloride [CoCl₂], manganese (II) formate [Mn(CHO₂)₂], cobalt (II) 2-ethylhexanoate [Co(C₈H₁₅O₂)₂], manganese (II) 2-ethylhexanoate [Mn(C₈H₁₅O₂)₂], dimanganese (0) decacarbonyl [Mn₂(CO)₁₀], iron (0) pentacarbonyl [Fe(CO)₅], triruthenium (0) dodecacarbonyl [Ru₃(CO)₁₂], an organophosphine of the type PR₃, PR₂H, or PRH₂, a ruthenium (I) dicarbonyl carboxylate [Ru(C_{w}HₓO_{y}N_{z})(CO)₂], wherein W, X, Y and Z are each whole numbers or zero, and a combination thereof.

9: The method of claim 1, wherein the first solvent comprises water.

10: The method of claim 1, wherein the catalyst is present in an amount of 0.1-10 mol % relative to the starting material.

11: The method of claim 1, wherein the concentration of the starting material is 1-10 M.

12: The method of claim 1, wherein the reaction mixture further comprises a polymerization inhibitor.

13: The method of claim 12, wherein the polymerization inhibitor is present in an amount of 1000-30,000 ppm.

14: The method of claim 12, wherein the polymerization inhibitor is one of hydroquinone, phenothiazine, methylene blue, 4-methoxyphenol, and a combination thereof.

15: The method of claim 1, wherein the reaction mixture further comprises a second solvent, the second solvent being an organic solvent.

16: The method of claim 15, wherein the second solvent is one of glyme, diglyme, triglyme, and tetraglyme.

17: The method of claim 1, further comprising:
after the predetermined reaction time has elapsed, separating produced organic acid from a residue remaining in the rest of the reaction mixture, the residue comprising unreacted starting material;
placing the unreacted starting material in a second reaction vessel with a solvent and a catalyst,
pressurizing the second reaction vessel to the predetermined pressure;
heating the second reaction vessel to the predetermined temperature; and
maintaining the predetermined temperature for the predetermined reaction time.

18: The method of claim 17, wherein the second reaction vessel is the same as the first reaction vessel.

19: The method of claim 1, further comprising:
while the predetermined temperature is maintained for the predetermined reaction time, maintaining a partial water vapor pressure at or below 540 psig.

20: A method for the selective production of an organic acid, comprising:
combining a starting material with a catalyst to form a reaction mixture;
exposing the reaction mixture to a predetermined pressure;
exposing the reaction mixture to a predetermined temperature; and
maintaining the predetermined temperature for a predetermined reaction time,
wherein the organic acid has a chemical formula of CH₂CRCOOH, wherein R is either a H or a CH₃, and
wherein the starting material is a at least one of a dicarboxylic acid, a tricarboxylic acid, and an anhydride of a dicarboxylic or tricarboxylic acid.
